(19) United States
(12) Patent Application Publication
(10) Pub. No.: US 2013/0150764 A1
(43) Pub. Date: Jun. 13, 2013

(54) Non-Adherent Wound Dressings and Related Methods Therefor

(71) Applicants: Harish A Patel, Norfolk, MA (US); Ajit K. Moghe, Bellingham, MA (US)

(72) Inventors: Harish A Patel, Norfolk, MA (US); Ajit K. Moghe, Bellingham, MA (US)

(73) Assignee: Tyco Healthcare Group LP, Mansfield, MA (US)

(21) Appl. No.: 13/316,028

(22) Filed: Dec. 9, 2011

(51) Int. Cl. A61F 13/00; B65B 55/00; B05D 3/00; B31B 1/26; B05D 7/00; D04H 1/46; B31B 1/14

(52) U.S. Cl. 602/44; 602/43; 602/48; 28/104; 28/169; 139/420 R; 53/431; 493/340; 493/395; 427/2.31

(57) ABSTRACT

A wound dressing is disclosed. The wound dressing includes an oil emulsion and a substrate including a plurality of first yarns and a plurality of second yarns, wherein the plurality of first yarns includes a plurality of first fibers comprising a cellulosic material and the plurality of second yarns includes a plurality of second fibers comprising a non-adherent polymeric material. In other cases, the substrate can be a woven substrate, a nonwoven substrate, or a knitted substrate of nonadherent polymeric fibers or yarns.

### FIELD

The present disclosure relates to substrates such as fabrics, and more particularly, to wound dressings including substrates formed with fibers impregnated with an oil emulsion or exhibit none or low adherency to wounds.

### BACKGROUND

Wound dressings have been used in the medical industry to protect and/or facilitate healing of open wounds. Wound dressings are generally placed over a wound to protect and promote healing of the wound. In the case of exuding wounds, such as pressure sores, ulcers, and burns, it is customary to provide a dressing having an absorbent material for absorbing at least a portion of the wound exudates as it is produced. Absorbing exudates promotes healing by removing potentially harmful bacteria from the wound bed, and also prevents damage to the surrounding skin that can be caused by an excessively moist environment. The absorbent material temporarily stores the excess exudates until removal thereof, typically periodically and replaced with a new dressing.

Woven gauze fabric has been used as a wound dressing to absorb wound exudates and to protect the wound from unwanted environmental factors. Such fabric is loosely woven and includes yarns made of cellulosic fibers, such as cotton and viscose rayon. The absorbency characteristics of the dressing depends on the material of construction. For example, the absorbency capacity of gauze fabrics depends on the absorbency of the constituent fibers in the yarn and the absorption capacity of the interstices within the yarn and between successive yarns.

Some absorbent materials utilized in some wound dressings, such as cotton, tend to become attached to a healing wound bed and may shed small fibers into the wound that may remain in the wound when the dressing is changed. Removing the dressing and/or stray fibers can be a labor intensive procedure that may further damage the wound, and neglecting to remove stray fibers may cause irritation or result in granuloma formation and otherwise inhibit natural healing of the wound.

### SUMMARY

One or more aspects of the invention can be directed to wound dressings. The wound dressing can comprise a substrate comprising a plurality of first yarns comprising cellulosic fibers, and a plurality of second yarns comprising non-adherent polymeric fibers; and an oil emulsion disposed on at least a portion the substrate. The plurality of first yarns, in some embodiments of the invention, can comprise heterogeneous yarns comprising a first cellulosic fiber and a second cellulosic fiber. The substrate, in some embodiments of the invention, can be a woven fabric including the plurality of first yarns and the plurality of second yarns respectively interwoven in a warp direction and in a weft direction. The substrate, in some embodiments of the invention, can be a woven fabric including the plurality of first yarns and the plurality of second yarns respectively interwoven in a weft direction and in a warp direction. The plurality of first yarns, in some embodiments of the invention, can comprise at least one of cotton and viscose rayon. In some embodiments of the invention, at least a portion of the plurality of second yarns can be comprised of a non-adherent polymeric material selected from the group consisting of polyethylene, polypropylene, polyfluoroethylene, polyfluoropropylene, polyfluoropolyethylene glycol, polytetrafluoroethylene, polyethylene terephthalate, polyethylene naphthalate, polytrimethylene terephthalate, polybutylene terephthalate, and combinations thereof. The cellulosic material, in some embodiments of the invention, can comprise about 5% to about 50% by weight of the substrate. The non-adherent polymeric fibers, in some embodiments of the invention, can comprise about 50% to about 95% by weight of the substrate. The wound dressing, in accordance with some embodiments of the invention, can further comprise at least one bacteriostatic agent disposed with the oil emulsion. The at least one bacteriostatic agent can be, in accordance some particular embodiments of the invention, bismuth tribromophenate.

One or more aspects of the invention can be directed to a wound dressing comprising a substrate comprised of a polymer selected from the group consisting of polyester, polypropylene, polyethylene, and polytetrafluoroethylene; and an oil emulsion impregnated into at least a portion of the substrate. The substrate, in accordance with some embodiments of the invention, can consist essentially of a weft knitted fabric with from about 5% to about 75% petrolatum by weight of the wound dressing. The substrate, in accordance with some embodiments of the invention, can consists essentially of a warp knitted fabric with from about 5% to about 75% of petrolatum by weight of the wound dressing. The substrate, in accordance with some embodiments of the invention, can further comprise from about 1% to about 5% of at least one of an antibacterial agent and a bacteriostatic agent, by weight of the wound dressing. The substrate, in accordance with some embodiments of the invention, can consist essentially of woven polyester with from about 5% to about 75% petrolatum by weight of the wound dressing.

One or more aspects of the invention can be directed to a method of preparing a wound dressing, comprising providing a substrate comprised of a non-adherent polymer selected from the group consisting of polyester, polypropylene, polyethylene, and polytetrafluoroethylene; introducing an oil emulsion into the substrate to produce the wound dressing; and sterilizing the wound dressing. In accordance with some embodiments of the invention, providing the substrate can comprise preparing a substrate consisting essentially of polyester, and introducing the oil emulsion can comprise impregnating petrolatum into the substrate to produce a wound dressing having from about 5 wt % to about 75 wt % petrolatum. In accordance with some embodiments of the invention, providing the substrate can comprise hydroentangling polyester fibers to produce a nonwoven substrate, and introducing the oil emulsion can comprise impregnating petrolatum into the nonwoven substrate to produce a wound dressing having from about 5 wt % to about 75 wt % petrolatum.

One or more aspects of the invention can be directed to methods of preparing a wound dressing. One or more further aspects of the invention can be directed to facilitating wound treatment. The method can comprise providing a fabric comprising cellulosic fibers woven with non-adherent polymeric fibers comprised of at least one polymer selected from the group consisting of polyester, polypropylene, polyethylene, and polytetrafluoroethylene; and impregnating an oil emulsion into the fabric to produce the wound dressing; and sterilizing the wound dressing. In some embodiments of the invention, providing the fabric comprises weaving the non-adherent polymeric fibers, in a warp direction, with the cellulosic fibers, in the weft direction, to produce a woven fabric with at least about 50% by weight of non-adherent polymeric fibers. In some further embodiments of the invention, providing the fabric comprises weaving the cellulosic fibers, in a warp direction, with the non-adherent polymeric fibers, in the weft direction, to produce a woven fabric with at least about 50% by weight of non-adherent polymeric fibers. In still further embodiments of the invention, providing the fabric comprises weaving at least a portion of the non-adherent polymeric fibers with at least a portion of the cellulosic fibers in the warp direction to produce a woven fabric with at least about 50% by weight of non-adherent polymeric fibers. In yet further embodiments of the invention, providing the fabric comprises weaving at least a portion of the non-adherent polymeric fibers and at least a portion of the cellulosic fibers in the weft direction to produce a woven fabric with at least about 50% by weight of non-adherent polymeric fibers. One or more embodiments of the invention can further comprise disposing the fabric into a sealable package, and wherein impregnating the oil emulsion comprises introducing the oil emulsion into the fabric disposed in the sealable package. In some embodiments of the invention, providing the fabric can comprise weaving the cellulosic fibers with the non-adherent polymeric fibers to produce a woven article; bleaching the woven article; tentering the bleached, woven article; cutting the bleached, woven article to produce a woven fabric; folding the woven fabric to produce a gauze or substrate. In some further embodiments of the invention, folding the woven fabric comprises folding the woven fabric to produce the dressing, gauze or substrate having any of three plies, four plies, five plies, six plies, eight plies, ten plies, twelve plies, sixteen plies, 24 plies, 32 plies, 48 plies, 50 plies, 144 plies, and 216 plies. One or more embodiments of the invention can further comprise introducing at least one bacteriostatic agent into the fabric. In some embodiments of the invention, the non-adherent polymeric fiber can be comprised of a material selected from the group consisting of polyethylene, polypropylene, polyfluoroethylene, polyfluoropropylene, polytetrafluoroethylene, polyethylene terephthalate, polyethylene naphthalate, polytrimethylene terephthalate, polybutylene terephthalate, and combinations thereof.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure:

FIGS. 1-5 are schematic illustrations showing various cross-sectional views of fibers that can be utilized as, for example, a wound dressing, in accordance with one or more embodiments of the invention;

FIG. 6 is schematic illustration showing a perspective view of a yarn of a wound dressing in accordance with one or more embodiments of the invention;

FIG. 7 is schematic illustration showing a perspective view of a yarn of a wound dressing in accordance with one or more embodiments of the invention; and

FIGS. 8-16 are schematic illustrations showing various of embodiments of wound dressings in accordance with one or more aspects of the invention.

### DETAILED DESCRIPTION

One or more aspects of the present invention can be directed to advantageously providing a wound dressing with customized properties, such as, for example, any one or more of increased strength, lower surface lint, lower adhesion to a wound, and increased fluid (exudates) transfer, to facilitate wound healing.

One or more aspects of the invention can be directed to wound dressings and methods for making the same. A wound dressing of the present disclosure includes a substrate having a plurality of first yarns fabricated, a plurality of second yarns fabricated from a non-adherent polymer material, and an oil emulsion disposed on and/or within the substrate. As used herein, the term “oil emulsion” includes soft paraffin, which is a semi-solid mixture at room temperature, of hydrocarbons with a carbon number of about twenty-five and above and, in embodiments, can be petrolatum, and can denote a composition including a mixture of hydrocarbons having a Chemical Abstracts Service Registry No. 8009-03-8.

One or more embodiments of wound dressings according to the present invention may be used in treating burn wounds and other wounds where non-adherent properties of the wound dressing are desirable. In one or more embodiments of the invention, wound dressings typically have enhanced non-adherent properties due to both the inclusion of an oil emulsion and non-adherent polymer materials.

Suitable materials from which the wound dressing may be formed may have the following characteristics: sufficiently strong to avoid tearing of portions thereof; sufficiently inert to avoid foreign body reactions when retained on or in the body for long periods of time; easily sterilized to prevent the introduction of infection when the dressing is placed upon or in the body; and suitable handling characteristics for placement in the desired location on the body. The wound dressing may also be sufficiently pliable to conform to a tissue surface, such as a wound, and flex with movement of the tissue.

In accordance with one or more aspects of the invention, the wound dressing can comprise a substrate comprising a plurality of first yarns comprising cellulosic fibers, and a plurality of second yarns comprising non-adherent polymeric fibers; and an oil emulsion disposed on at least a portion the substrate. In accordance with some embodiments of the invention, the first yarns can comprise at least one of a bast fiber and another cellulosic fiber, and the second yarns can comprise non-adherent polymeric fibers; and an oil emulsion disposed on at least a portion the substrate. In accordance with further embodiments of the invention, the wound dressing can consist of first yarns of at least one of a bast fiber and a cellulosic fiber, and second yarns comprising non-adherent polymeric fibers; and an oil emulsion disposed on at least a portion the substrate. In accordance with still further embodiments of the invention, the wound dressing can consist essentially of first yarns consisting essentially of at least one of a bast fiber and a cellulosic fiber, and second yarns consisting essentially of non-adherent polymeric fibers; and an oil emulsion disposed on at least a portion the substrate. In accordance with yet further embodiments of the invention, the wound dressing can consist of first yarns consisting of at least one of a bast fiber and a cellulosic fiber, and second yarns consisting of non-adherent polymeric fibers; and an oil emulsion disposed on at least a portion the substrate. The substrate, in some embodiments of the invention, can be a woven fabric including the first yarns and the second yarns respectively interwoven in a warp direction and in a weft direction. The substrate, in some embodiments of the invention, can be a woven fabric including the first yarns and the second yarns respectively interwoven in a weft direction and in a warp direction.

The first yarns, in some embodiments of the invention, can be heterogeneous yarns comprising a first cellulosic fiber and a second cellulosic fiber. The first yarns, in some embodiments of the invention, can be heterogeneous yarns consisting essentially of a first cellulosic fiber and a second cellulosic fiber. The heterogeneous yarns, in some embodiments of the invention, can consist of a first cellulosic fiber and a second cellulosic fiber. The first yarns, in some embodiments of the invention, can comprise heterogeneous yarns comprising a cellulosic material and a bast fiber. The first yarns, in some embodiments of the invention, can consist essentially of a cellulosic fiber and a bast fiber. The first yarns, in some embodiments of the invention, can consist of a cellulosic fiber and a bast fiber. The first yarns, in accordance with some embodiments of the invention, can consist essentially of bast fibers. In accordance with further embodiments of the invention, the first yarns can consist of bast fibers. The first yarns, in some embodiments of the invention, can comprise at least one of cotton and viscose rayon.

In some embodiments of the invention, at least a portion of the plurality of second yarns can be comprised of a non-adherent polymeric material selected from the group consisting of polyethylene, polypropylene, polyfluoroethylene, polyfluoropropylene, polyfluoropolyethylene glycol, polytetrafluoroethylene, polyethylene terephthalate, polyethylene naphthalate, polytrimethylene terephthalate, polybutylene terephthalate, and combinations thereof. The cellulosic material, in some embodiments of the invention, can comprise about 5% to about 50% by weight of the substrate. The non-adherent polymeric fibers, in some embodiments of the invention, can comprise about 50% to about 95% by weight of the substrate. The wound dressing, in accordance with one or more embodiments of the invention, can further comprise at least one bacteriostatic agent, such as with the oil emulsion. The at least one bacteriostatic agent can be bismuth tribromophenate. The wound dressing, in accordance with one or more embodiments of the invention, can further comprise at least one antibacterial agent, such as with the oil emulstion. The at least one antibacterial agent can be polyhexamethylene biguanide.

One or more aspects of the invention can be directed to a wound dressing comprising a knitted substrate comprising a polyester yarn; and an oil emulsion disposed on at least a portion of the knitted substrate. In accordance with some embodiments of the invention, the wound dressing can comprise a knitted substrate consisting essentially of a polyester yarn, and an oil emulsion disposed on at least a portion of the knitted substrate. In accordance with some embodiments of the invention, the wound dressing can comprise a knitted substrate consisting of a polyester yarn, and an oil emulsion disposed on at least a portion of the knitted substrate. In some embodiments of the invention, the substrate can comprise a weft knitted fabric with from about 5 wt % to about 75 wt % of petrolatum. In some embodiments of the invention, the substrate comprises a warp knitted fabric with from about 5 wt % to about 75 wt % of petrolatum. The wound dressing, in accordance with some embodiments of the invention, can comprise from about 1 wt % to about 5 wt % of at least one of an antibacterial agent and a bacteriostatic agent.

One or more aspects of the invention can be directed to a wound dressing consisting essentially of a knitted substrate of polyester yarn and from about 5 wt % to about 75 wt % of petrolatum. One or more aspects of the invention can be directed to a wound dressing consisting of a knitted substrate of polyester yarn and from about 5 wt % to about 75 wt % of petrolatum.

One or more aspects of the invention can be directed to a wound dressing comprising a substrate comprised of a polymer that is non-adherent to wound surfaces; and an oil emulsion impregnated into at least a portion of the substrate. One or more further aspects of the invention can be directed to a wound dressing consisting essentially of a substrate of a polymer that is non-adherent to wound surfaces; and an oil emulsion impregnated into at least a portion of the substrate. One or more still further aspects of the invention can be directed to a wound dressing consisting of a substrate of a polymer that is non-adherent to wound surfaces; and an oil emulsion impregnated into at least a portion of the substrate. The substrate, in accordance with some embodiments of the invention, can consist essentially of woven polyester fibers or yarns with from about 5% to about 75% petrolatum, by weight of the wound dressing. The substrate, in accordance with further embodiments of the invention, can consist essentially of nonwoven polyester fibers or yarns with from about 5% to about 75% petrolatum, by weight of the wound dressing. The non-adherent polymer can be a material selected from the group consisting of polyethylene, polypropylene, polyfluoroethylene, polyfluoropropylene, polyfluoropolyethylene glycol, polytetrafluoroethylene, polyethylene terephthalate, polyethylene naphthalate, polytrimethylene terephthalate, and polybutylene terephthalate. In particular embodiments of the invention, the substrate consists of a combination of two or more of the non-adherent polymers.

In one or more aspects of the invention, preparing a wound dressing can comprise providing a fabric comprising cellulosic fibers woven with non-adherent polymeric fibers comprised of at least one polymer selected from the group consisting of polyester, polypropylene, polyethylene, and polytetrafluoroethylene; and impregnating an oil emulsion into the fabric to produce the wound dressing; and sterilizing the wound dressing. In some embodiments of the invention, providing the fabric comprises weaving the non-adherent polymeric fibers, in a warp direction, with the cellulosic fibers, in the weft direction, to produce a woven fabric with at least about 50% by weight of non-adherent polymeric fibers. In some further embodiments of the invention, providing the fabric comprises weaving the cellulosic fibers, in a warp direction, with the non-adherent polymeric fibers, in the weft direction, to produce a woven fabric with at least about 50% by weight of non-adherent polymeric fibers. In still further embodiments of the invention, providing the fabric comprises weaving at least a portion of the non-adherent polymeric fibers with at least a portion of the cellulosic fibers in the warp direction to produce a woven fabric with at least about 50% by weight of non-adherent polymeric fibers. In yet further embodiments of the invention, providing the fabric comprises weaving at least a portion of the non-adherent polymeric fibers and at least a portion of the cellulosic fibers in the weft direction to produce a woven fabric with at least about 50% by weight of non-adherent polymeric fibers. One or more embodiments of the invention can further comprise disposing the fabric into a sealable package, and wherein impregnating the oil emulsion comprises introducing petrolatum into the fabric disposed in the sealable package. In some embodiments of the invention, providing the fabric can comprise weaving the at least one of cellulosic fibers and bast fibers with the non-adherent polymeric fibers to produce a woven article; bleaching the woven article; tentering the bleached, woven article; cutting the bleached, woven article to produce a woven fabric; folding the woven fabric to produce a gauze. In some further embodiments of the invention, folding the woven fabric comprises folding the woven fabric to produce the gauze having any of three plies, four plies, five plies, six plies, eight plies, ten plies, twelve plies, sixteen plies, 24 plies, 32 plies, 48 plies, 50 plies, 144 plies, and 216 plies. One or more embodiments of the invention can further comprise introducing at least one bacteriostatic agent into the fabric. One or more embodiments of the invention can further comprise introducing at least one antibacterial agent into the fabric. In some embodiments of the invention, the non-adherent polymeric fiber can be comprised of a material selected from the group consisting of polyethylene, polypropylene, polyfluoroethylene, polyfluoropropylene, polytetrafluoroethylene, polyethylene terephthalate, polyethylene naphthalate, polytrimethylene terephthalate, polybutylene terephthalate, and combinations thereof. In accordance with one or more aspects of the invention, facilitating wound treatment can comprise providing a fabric comprising cellulosic fibers woven with non-adherent polymeric fibers comprised of at least one polymer selected from the group consisting of polyester, polypropylene, polyethylene, and polytetrafluoroethylene; and impregnating an oil emulsion into the fabric to produce the wound dressing; and sterilizing the wound dressing.

The first yarns can comprise, but are not limited to, naturally occurring cellulosic materials as well as synthetically-modified and/or regenerated cellulose materials. Synthetically-modified and/or regenerated cellulosic materials include cellulose and polysaccharide derivatives, including alkyl celluloses, hydroxyalkyl celluloses, cellulose ethers, cellulose esters, nitrocelluloses, and chitosan. Specific examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose (CMC), cellulose triacetate, and cellulose sulfate sodium salt. These may be collectively referred to herein, in embodiments, as “celluloses.” Additional synthetic cellulosic materials include, but are not limited to, rayon, rayon acetate, viscose rayon, and lyocell. Natural cellulosic materials that may be utilized in any one or more configurations of the invention include, for example, cotton, linen, combinations, and derivatives thereof. Other materials that may be utilized in any one or more configurations of the invention include, for example, bast fibers or other fibers derived from plant stems or barks such as, for example, flax, hemp, jute, ramie, and derivatives thereof. Other materials can include man-made cellulosic materials such as, for example, rayon, rayon acetate, viscose rayon, lyocell, and combinations thereof. Synthetically modified natural polymers of cellulose derivatives may be utilized in any one or more configurations of the invention include, for example, alkyl celluloses, hydroxyalkyl celluloses, cellulose ethers, cellulose esters, nitrocelluloses, and chitosan. Non-limiting examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt. Any of the substrates can incorporate one or more types of fibers. For example, one or more embodiments of the invention can comprise any of cotton, linen, with any one or more bast fibers such as any of flax, hemp, jute, and ramie. Commercially available bast fibers that can be utilized in the various embodiments of the invention include those available as, for example, CRAILAR fibers from Naturally Advanced Technologies Inc., Victoria, British Columbia, Canada.

Suitable non-adherent polymeric materials that can be utilized in any of the substrates disclosed herein can include, but are not limited to, polyolefins such as polyethylene and polypropylene including atactic, isotactic, syndiotactic, and blends thereof; polyethylene oxides; ultra high molecular weight polyethylene; copolymers of polyethylene and polypropylene; polyisobutylene and ethylene-alpha olefin copolymers; fluorinated polyolefins such as fluoroethylenes, fluoropropylenes, fluoroPEGs, and polytetrafluoroethylene; polyamides such as nylon and polycaprolactam; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; polychlorofluoroethylene; polyacrylonitrile; polyaryletherketones; polyvinyl ketones; copolymers of vinyl monomers with each other and olefins; acrylonitrile-styrene copolymers; polyimides; aramids; rayon; rayon-triacetate; and copolymers and combinations thereof. In other cases, the non-adherent fibers that can be utilized in any of the substrates disclosed herein can be comprised of sodium alginate, calcium alginate, or combinations thereof.

Thus, for example, some aspects of the present invention can be directed to embodiments of wound dressings comprising at least one woven, nonwoven, or knitted substrate of alginate fibers and petrolatum. In other embodiments, however, the wound dressing can consist essentially of any of a woven substrate of alginate fibers, a nonwoven substrate of alginate fibers, and a knitted substrate of alginate fibers; and petrolatum. In other embodiments, however, the wound dressing can consist of any of a woven substrate of alginate fibers, a nonwoven substrate of alginate fibers, and a knitted substrate of alginate fibers; and petrolatum.

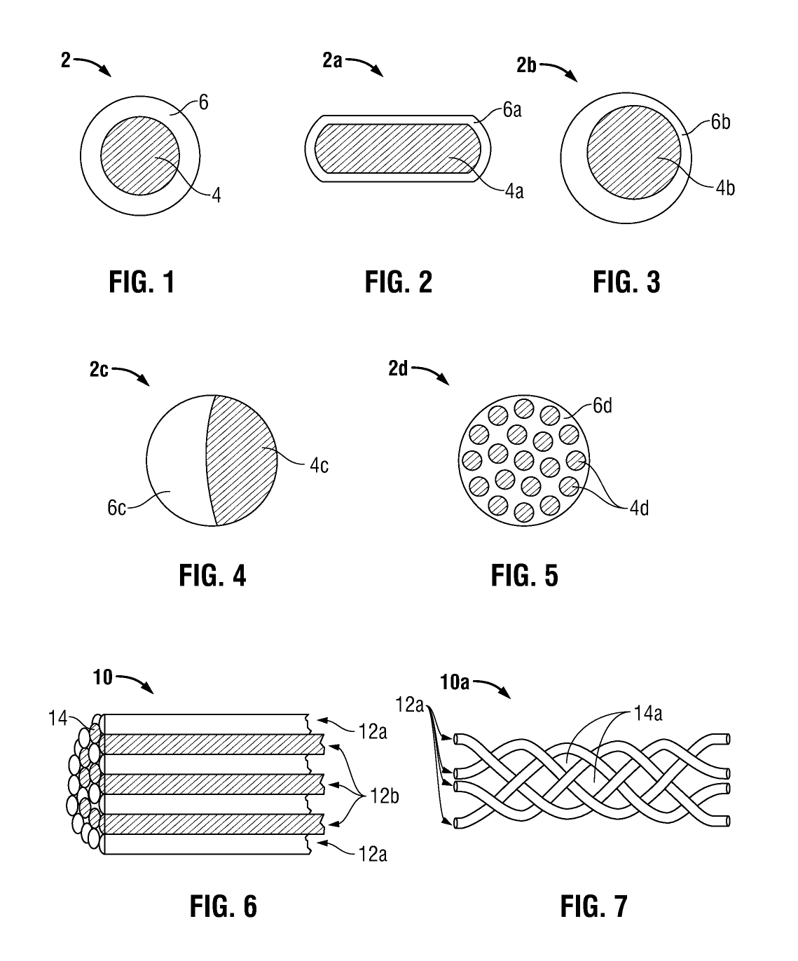

The wound dressing can comprise bi-component, monofilament, or multifilament fibers formed from cellulosic and/or non-adherent polymeric materials. Bi-component fiber are typically fibers of two polymers which may have different chemical and/or physical properties. Bi-component fibers may include cellulosic material in an amount from 5% to about 50% by weight of the fibers, in embodiments from about 10% to about 45% by weight of the fibers, and in accordance with some further embodiments of the invention, from about 15% to about 40% by weight of the fibers. The bi-component fibers may include the non-adherent polymeric material described herein from 50% to about 95% by weight of the fibers, in embodiments from about 55% to about 90% by weight of the fiber, and in further embodiments from about 60% to about 80% by weight of the fibers. As illustrated in FIG. 1, bi-component fiber 2 can have a core polymer 4 and a sheath polymer 6. Core polymer 4 may be fabricated from a first polymeric material and sheath polymer 6 may be fabricated from a second polymeric material having different characteristics than the first polymeric material.

Bi-component fiber 2 may be a monofilament fiber which is, for example, co-extruded from two distinct polymers to exhibit a concentric sheath-core arrangement. Fiber 2 can have a round cross-sectional profile including a core polymer 4 surrounded by a sheath polymer 6. The core polymer 4 and sheath polymer 6 can be concentrically arranged. In any one or more configuration of the various fibers may involve various cross-sectional shapes, such as a flattened cross-sectional shape as exemplarily illustrated by fiber 2a in FIG. 2, as well as other modified cross-sections configurations which may be co-extruded to generate fibers with more complex profiles. As illustrated in FIG. 3, bi-component fiber 2b may also exhibit an eccentric (e.g., offset center) sheath-core arrangement. Fiber 2b can have an off-center core polymer 4b surrounded by sheath polymer 6b. FIG. 4 illustrates a bi-component fiber 2c similar to fibers 2, 2a, 2b, but differs in that core polymer 4c and sheath polymer 6c each can occupy a portion of the outer surface of the fiber 2c.

As exemplarily illustrated in FIG. 5, bi-component fiber 2d may also be a multifilament fiber which may be spun and processed from microdenier filaments of core polymer 4d. Fiber 2d can exhibits an islands-in-the-sea arrangement where two or more “islands,” or core polymer filaments 4d are surrounded by a “sea,” or sheath polymer 6d. This arrangement may provide for very fine strands of island polymer filaments 4d to be effectively handled by manufacturing equipment to spin and form fiber 2d. Core polymer filaments 4d may be arranged so as to be generally non-intersecting along their length.

Although not necessarily parallel, core polymer filaments 4d may be generally free from entanglement or interlacing over a substantial portion of their length. Alternatively, core polymer filaments 4d may be woven, braided, or entangled by various processes within the purview of those skilled in the art. The core polymer filaments 4d may be spun inside the sheath polymer 6d. The number of polymer filaments 4d formed within the fiber 2d may be from about two to about fifty, in embodiments may be from about ten to about forty.

The sheath polymer may be applied to the core polymer of the bi-component fiber by, for example, extrusion, co-extrusion, pultrusion, gel spinning with one of the aforementioned processes, melt coating, spray coating, ultrasonic spray coating, electrostatic coating, powder coating, solvent/immersion coating such as dipping, spraying, solvent evaporation, sheath heat crimping, chemical surface modification, and combinations thereof.

The surface of the core polymer may be porous to facilitate anchoring or impregnating at least a portion of the sheath polymer into the core. Any of the fibers can have a tailored or predetermined porosity which may be achieved by roughening the surface of the core polymer. Alternatively, the core polymer may have a smooth, non-porous surface such that the core and sheath polymers have little or no adhesion to each other.

In accordance with some embodiments of the invention, the core of the bi-component fiber can provide strength and maintain the integrity of the entire bi-component fiber, while the sheath can provide a smooth, non-adherent outer surface which can be advantageous in promoting non-adherence when displaced against tissue.

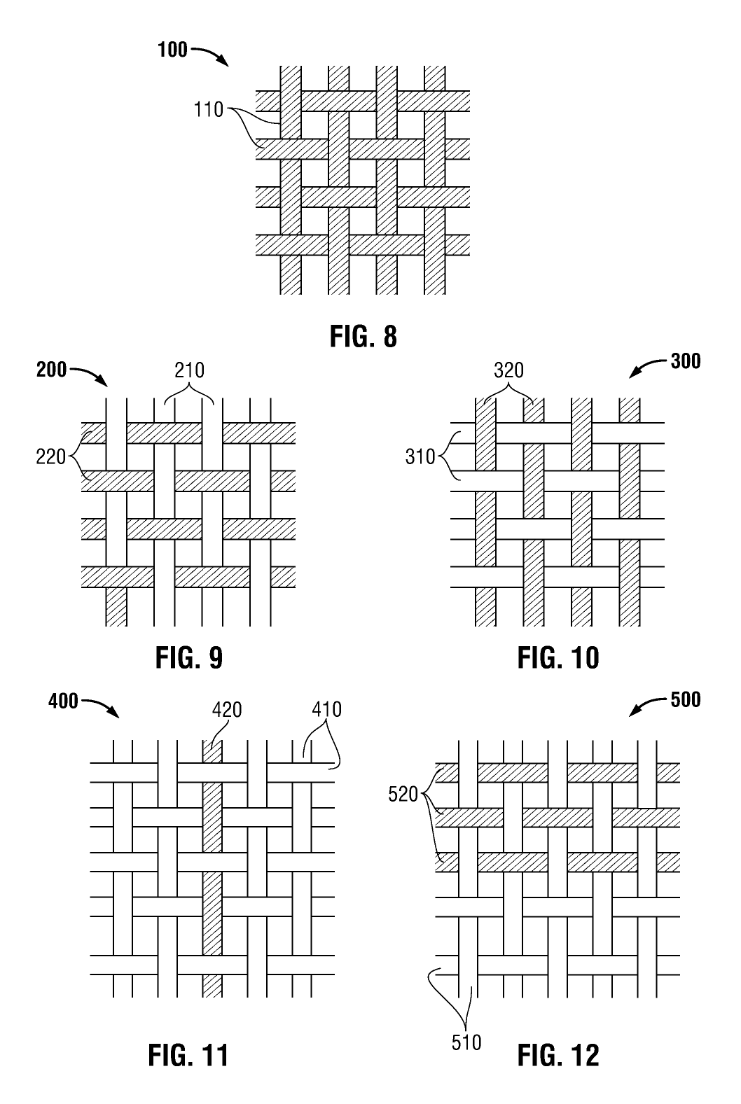

FIGS. 7 and 8 exemplarily illustrate embodiments in accordance with some aspects the invention that utilize multifilament yarns. Two or more filaments may be used to form the multifilament yarns. The filament may be arranged to create openings therebetween and the yarns may also be arranged relative to each other to form openings in the wound dressing. The spacing between the yarns may vary depending on the medical or surgical application and desired wound dressing properties. Any of the wound dressings in accordance with any one or more aspects of the invention may be of any suitable size.

Multifilament yarns may be heterogeneous or homogeneous yarns. As illustrated in FIG. 6, heterogeneous yarns 10 can be configured to include at least two dissimilar filaments 12a and 12b and include openings 14 formed between filaments 12a and 12b. Yarns 10, such as, filaments 12a and 12b, may be formed from cellulosic and non-adherent polymeric materials.

Homogeneous yarns 10a, as shown in FIG. 7, can be configured to include at least two substantially similar filaments 12a, and can also include openings 14a formed between filaments 12a. In embodiments in which at least two filaments form a yarn, the filaments may be drawn (FIG. 6), braided (FIG. 7), or otherwise oriented, crinkled, twisted, commingled or air entangled to form the yarn.

Yarns may include any number of fibers and be dimensioned in a variety of sizes and shapes. Yarns may have a size ranging from about 25 English cotton yarn number (Ne) count to about 40 Ne count, in embodiments from about 30 Ne to about 37 Ne. Yarns may have a break factor from about 1,700 pound cotton count (lb Ne) to about 2,500 lb Ne, in embodiments from about 2,000 lb Ne to about 2,200 lb Ne.

The yarns may be braided, twisted, aligned, fused, or otherwise joined to form a variety of different wound dressing configurations. The yarns may be woven, knitted, interlaced, braided, or combinations thereof, to be formed into a substrate, such as a fabric, for a wound dressing or by other non-weaving techniques. The structure thereof will vary depending upon the assembling technique utilized to form the fabric, as well as other factors such as the type of fibers used, the tension at which the yarns are held, and the mechanical properties required of the wound dressing.

In some embodiments in accordance with some aspects of the invention, knitting may be utilized to form any of the various wound dressings. Knitting typically involves the intermeshing of yarns to form loops, or inter-looping of the yarns. In some embodiments of the invention, any of the various herein disclosed yarns may be warp-knitted thereby creating vertical interlocking loop chains and/or may be weft-knitted thereby creating rows of interlocking loop stitches across the wound dressing.

Any of the substrates of the present invention may be formed into a nonwoven substrate by any technique including any of mechanically, chemically, thermally bonding the yarns into a sheet or web in a random or systematic arrangement. For example, one or more yarns of the present invention may be mechanically bound by entangling the yarns to form the wound dressing by means other than knitting or weaving, such as matting, pressing, stitch-bonding, needlepunching, or otherwise interlocking the yarns to form a binderless network. Alternatively, any of the yarns may be chemically bound by an adhesive, such as a hot melt adhesive, or be thermally bound by a binder such as a powder, paste, or melt, and melting the binder on the sheet or web of yarns.

In other cases, any of the substrates of the present invention may be formed by spunlacing or hydroentangling fiber or yarns that have been formed by carding, airlaying, or wetforming processes, and striking the yarns or fibers with high speed jets of water to at least partially entangle at least a portion of the yarn or fiber, with itself and/or with other yarns or fibers. In still other cases, any of the nonwoven substrates of the present invention may be formed by needlepunching a precursor web of fibers or yarns, which typically have been prepared by spunbonding or by carding, and striking the yarns or fibers with barbed felting needles to at least partially interlock at least a portion of the yarn or fiber, with itself and/or with other yarns or fibers. In yet other cases, any of the nonwoven substrates of the present invention may be formed by extruding molten polymeric material into filaments, overlaying the molten filaments and allowing the filaments to cool and form bonds at contact points. In further cases, any of the substrates of the invention can be formed by meltblowing techniques which typically involve extruding molten polymeric material and drawing the extruded molten filaments with high velocity jets of air to form fine filaments that have one or more bond contact points. In yet further cases, any of the substrates of the invention may be formed by preparing a precursor web with thermoplastic polymeric material, which typically can be formed by any of carding, airlaying, or spunbonding, and melting at least a portion of the thermoplastic material, typically by utilizing heated calender rolls, to form bonds with other fibers. In yet further cases, any of the substrates of the present invention can be formed by chemically bonding at least a portion of fibers in the substrate by utilizing a chemical binder, such as latex.

Weaving may be utilized to form any of the substrates or wound dressings of the invention. Weaving may involve, for example, the intersection of two sets of straight yarns, warp and weft, which cross and interweave at right angles to each other, or the interlacing of two yarns at right angles to each other. The yarns may be arranged to form a net wound dressing which has isotropic or near isotropic tensile strength and elasticity.

Yarns described above may include any number and combination of multifilament, monofilament, and/or bi-components fibers formed from cellulosic or non-adherent polymeric materials. Cellulosic material may be present in an amount from 5% to about 50% by weight of the yarns, in embodiments from about 10% to about 45% by weight of the yarns, and in further embodiments from about 15% to about 40% by weight of the yarns. The yarns may include the non-adherent polymeric material described above from 50% to about 95% by weight of the yarns, in embodiments from about 55% to about 90% by weight of the fiber, and in further embodiments from about 60% to about 80% by weight of the yarns.

As illustrated in FIG. 8, substrate or wound dressing 100 can include yarns 110 including fibers of cellulosic and non-adherent polymeric materials. Yarns 110 may be monofilament or multifilament, homogeneous or heterogeneous yarns, as described herein. While illustrated as being woven, the yarns 110 may be interconnected in any manner as described herein. For example, yarns in staple form may be spun using standard spinning methodologies, such as open end spinning, ring spinning, air jet spinning, and other techniques to form any of the substrates.

As shown in FIG. 9, substrate or wound dressing 200 can include first yarns 210 including cellulosic fibers arranged in a warp direction and second yarns 220 including non-adherent polymeric fibers interlaced between the first yarns 210 in a weft direction to form a weaved pattern. In a further embodiment, as shown in FIG. 10, substrate or wound dressing 300 may include first yarns 310 including cellulosic fibers arranged in a weft direction and second yarns 220 including non-adherent polymeric fibers in a warp direction.

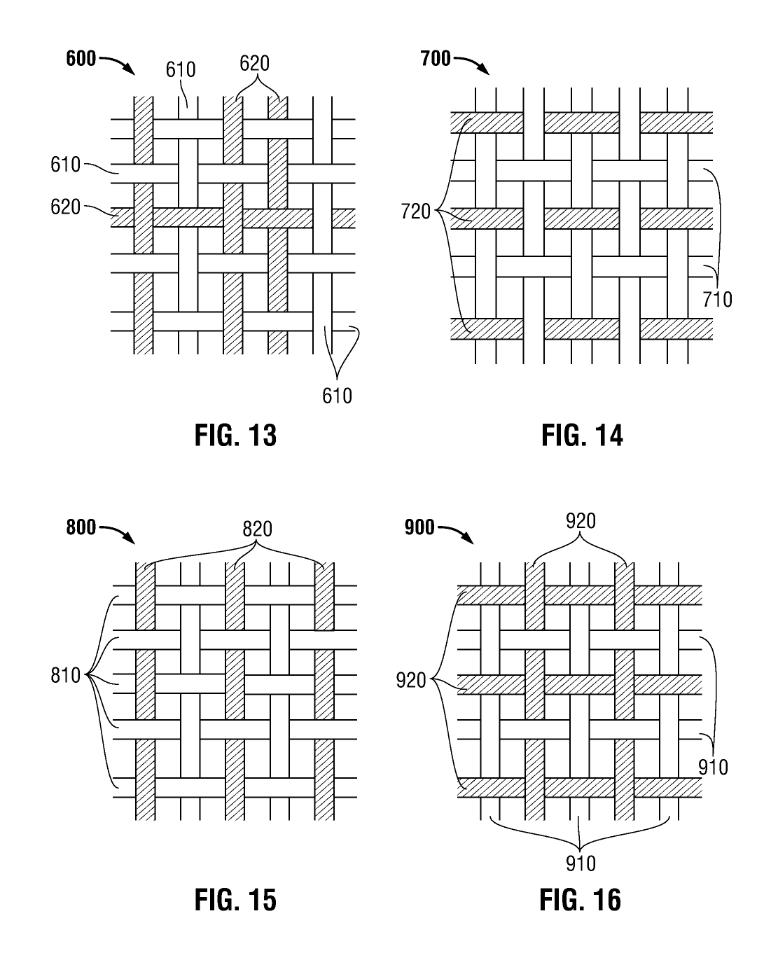

In other embodiments, as illustrated in FIGS. 11-13, any of substrates or wound dressings 400, 500, and 600 may include at least one yarn 420 including non-adherent polymeric fibers in a warp direction (FIG. 11), at least one yarn 520 including non-adherent polymeric fibers in a weft direction (FIG. 12), or at least one yarn 620 including non-adherent polymeric fibers in both warp and weft directions (FIG. 13). The remainder of the yarns 410, 510, and 610 include cellulosic fibers.

FIGS. 14-16 illustrate substrates or wound dressings 700, 800, and 900 including alternating yarns of cellulosic and polymer fibers. As shown in FIG. 14, yarns 710 can comprise cellulosic fibers and yarns 720 can comprise non-adherent polymeric fibers alternate in a weft direction, with fibers 710 including cellulosic fibers making up the yarns in the warp direction. FIG. 15 illustrates yarns 810 including cellulosic fibers and yarns 820 including non-adherent polymeric fibers alternating in a warp direction, with yarns 810 including cellulosic fibers making up the yarns in the weft direction. FIG. 16 illustrates yarns 910 including cellulosic fibers and yarns 920 including polymer fibers alternating in both warp and weft directions.

Wound dressings may include any number and combination of yarns formed from multifilament, monofilament, and/or bi-component fibers, which are formed from cellulosic or non-adherent polymeric materials. Cellulosic material may be present in an amount from 5% to about 50% by weight of the wound dressings, in embodiments from about 10% to about 45% by weight of the wound dressings, and in further embodiments from about 15% to about 40% by weight of the wound dressings. The wound dressings may include the non-adherent polymeric material described above from 50% to about 95% by weight of the wound dressings, in embodiments from about 55% to about 90% by weight of the fiber, and in further embodiments from about 60% to about 80% by weight of the wound dressings. In further embodiments, the wound dressing according to the present disclosure about 50% or more by weight of non-adherent polymeric fibers.

The fabric for fabricating the dressing, once formed, e.g., by weaving or knitting, may be bleached and may optionally be sterilized. Thereafter, the fabric may be tentered, e.g., setting warp and weft of the fabric at substantially right angles with respect to each other and stretching the yarns. The fabric can then be dried and cut into desired size. The cut portions of the fabric may then be folded to produce the substrate. Particular embodiments of the invention can involve embodiments comprising an absorbent gauze comprising cotton, with not more than about 55% by weight of rayon, as a plain woven cloth. Preferably, the absorbent gauze is sterile. Any of the various embodiments of the invention can involve substrates, such as an absorbent gauze, comprising warp threads in a range of from about 41 to about 47 per centimeter, filling threads in a range of 33 to 39 per centimeter. Any of the various embodiments of the invention can involve substrates, such as an absorbent gauze, with an average thread count in a range of about 76 to about 84 threads per 6.45 cm², and basis weight in a range of from about 43.8 to about 55.8 grams per square meter. Any of the various embodiments of the invention can involve substrates, such as an absorbent gauze, comprising warp threads in a range of from about 18 to about 22 per centimeter, filling threads in a range of 8 to 14 per centimeter. Any of the various embodiments of the invention can involve substrates, such as an absorbent gauze, with an average thread count in a range of about 27 to about 35 threads per 6.45 cm², and basis weight in a range of from about 18.1 to about 23.1 grams per square meter. Any of the various embodiments of the invention can involve substrates, such as an absorbent gauze, comprising warp threads in a range of from about 12 to about 16 per centimeter, filling threads in a range of 8 to 12 per centimeter. Any of the various embodiments of the invention can involve substrates, such as an absorbent gauze, with an average thread count in a range of about 21 to about 27 threads per 6.45 cm², and basis weight in a range of from about 12.1 to about 15.5 grams per square meter.

It is envisioned that any number of yarns, in various arrangements and patterns, may be used to form the substrates and wound dressings of the present invention. The yarns, fabrics, or substrates may be scoured and bleached to meet desirable, suggested, and/or mandated standards, such as USP gauze fabric standards. The yarns, fabrics, substrates or wound dressings may be sterilized using standard sterility protocols to conform to suggested or mandated sterility standards. For example, the various embodiments or components thereof of the invention can be sterilized to conform with sterilization standards of medical devices as set forth by the International Organization for Standardization including, for example, any of ISO 11135 for ethylene oxide sterilization for medical devices, ISO 11137 for gamma and e-beam sterilization for medical devices, and ISO 17665 for steam sterilization for medical devices. Sterilizing any of the yarns, substrates, gauze and wound dressings of the invention can involve any suitable technique that provides a desired level of sterility, such as a desired sterility assurance level, including, for example, any one or more of physical processes such as steaming, autoclaving, heating, chemical processes such as exposure to agents such as hydrogen peroxide, ethylene oxide, ozone, silver ions, or other oxidizing compounds such as sodium hypochlorite, irradiation processes such as exposure to gamma rays, electron beams, ultraviolet light and x-ray energy, and combinations thereof.

The fabricated substrate, e.g., gauze, may be impregnated or otherwise treated with an oil emulsion to produce the wound dressing. In accordance with embodiments of the invention, petrolatum may be applied to the substrate, such as gauze, after being disposed in a sealable package, e.g., prior to the package is sealed. Petrolatum may thus be selectively disposed on the wound-contacting surface of the wound dressing. In some cases, the oil emulsion may be applied to the entire wound dressing to fully impregnate all layers of the substrate. The amount of the oil emulsion present in the dressing may be from about 5% to about 75% by weight of the wound dressing, in embodiments may be from about 25% to about 50% by weight of the wound dressing. The substrate, or dressing, can then be sterilized, such as by exposure to steam in accordance with ISO 17665. Sterilization can be performed before or after introducing the oil emulsion in the package.

Bioactive agents such as polyhexamethylene biguanide, bismuth tribromophenate, or other medicaments, antimicrobial agents, bacteriostatic agents, hemostatic agents, tissue scaffolding agents, anti-thrombogenic agents, vasodilation agents, anesthetic agents, anti-inflammatory agents, anticancer agents, angiostatic agents, immune boosting agents, skin sealing agents, wound healing agents, and/or wound debriding agents, may be used to, for example, decrease the incidence of infection or otherwise promote healing of a wound. Other agents include those used in slow release treatments wherein the agent is released from a fiber or yarn into the wound over a period of time. Other bioactive agents that may utilized in any one or more variant embodiments of the invention can include, for example, therapeutic agents, organoleptic agents, and pharmaceutical agents. Any of the one or more bioactive agents may be disposed into any the fibers, yarns, substrates, and wound dressings of the invention by immersion thereof in a solution including the one or more agents, and, optionally, drying the solvents from the immersed, coated, infused fiber, yarn, substrate or wound dressing to any desired bioactive agent concentration, for example, to a concentration that at least partially inhibits any microbial activity therein. Introduction of the one or more bioactive agents can be performed during or after any one or more yarns fabrication, substrates fabrication, or wound dressing fabrication, or, in some cases, after any one of bleaching, and sterilizing. Further aspects of the invention can be directed to methods or techniques utilizing the substrates and gauze as wound dressings as disclosed herein to absorb wound exudates, to protect wounds, to cushion wound sites. Such methods and techniques can involve securing any of the wound dressings on the wound or wound site, replacing the wound dressing, and/or reapplying the wound dressing comprising, consisting essentially of, or consisting of the first and second fibers. The bioactive agent can be in an amount ranging from about 1 wt % to about 5 wt %, based on the weight of the dressing.

It should be understood that the wound dressings of the present disclosure are not limited to those illustrated and described herein and alternate wound dressings and components thereof may be utilized. Moreover, wound dressings of the present invention may be formed by layering one or more of the same or different wound dressings together to form a three-dimensional structure with any one or more desired dressing properties. For example, any of the layers of structure can utilize any of a substrate formed of woven fibers or yarns, a substrate formed of nonwoven fibers or yarns, and a substrate formed of knitted fibers or yarns.

Various modifications and variations of the polymers utilized in the wound dressing, as well as configurations of the fibers and yarns of the wound dressing will be apparent to those skilled in the art from the foregoing detailed description. Such modifications and variations are intended to come within the scope and spirit of the claims appended hereto. While several embodiments of the invention have been described, it is not intended that the disclosure be limited thereto, as it is intended that the disclosure be as broad in scope as the art will allow and that the specification be read likewise. Therefore, the above description should not be construed as limiting, but merely as exemplifications of embodiments of the present invention.

What is claimed is:

1. A wound dressing comprising a substrate comprising a plurality of first yarns comprising a cellulosic fiber, and a plurality of second yarns comprising non-adherent polymeric fibers; and an oil emulsion disposed on at least a portion the substrate.

2. The wound dressing of claim 1, wherein the substrate is a woven fabric including the plurality of first yarns and the plurality of second yarns respectively interwoven in a warp direction and in a weft direction.

3. The wound dressing of claim 1, wherein at least a portion of the plurality of second yarns is comprised of a non-adherent polymeric material selected from the group consisting of polyethylene, polypropylene, polyfluoroethylene, polyfluoropropylene, polyfluoropolyethylene glycol, polytetrafluoroethylene, polyethylene terephthalate, polyethylene naphthalate, polytrimethylene terephthalate, polybutylene terephthalate, and combinations thereof.

4. The wound dressing of claim 1, wherein the cellulosic material comprises about 5% to about 50% by weight of the substrate.

5. The wound dressing of claim 1, wherein the non-adherent polymeric fibers comprises about 50% to about 95% by weight of the substrate.

6. The wound dressing of claim 1, further comprising at least one a bacteriostatic agent and an antibacterial agent.

7. The wound dressing of claim 1, further comprising about 3 wt % bismuth tribromophenate, based on the weight of the wound dressing.

8. A wound dressing comprising a substrate comprised of a polymer selected from the group consisting of polyester, polypropylene, polyethylene, and polytetrafluoroethylene; and an oil emulsion impregnated into at least a portion of the substrate.

9. The wound dressing of claim 8, wherein the substrate consists essentially of a weft knitted fabric with from about 5 wt % to about 75 wt % petrolatum.

10. The wound dressing of claim 8, wherein the substrate consists essentially of a warp knitted fabric with from about 5 wt % to about 75 wt % petrolatum.

11. The wound dressing of claim 10, further comprising from about 1 wt % to about 5 wt % of at least one of an antibacterial agent and a bacteriostatic agent.

12. The wound dressing of claim 8, wherein the substrate consists essentially of a woven polyester with from about 5 wt % to about 75 wt % petrolatum.

13. A method of preparing a wound dressing, comprising:
providing a substrate comprised of a non-adherent polymer selected from the group consisting of polyester, polypropylene, polyethylene, and polytetrafluoroethylene;
introducing an oil emulsion into the substrate to produce the wound dressing; and
sterilizing the wound dressing.

14. The method of claim 13, wherein providing the substrate comprises preparing a substrate consisting essentially of polyester, and wherein introducing the oil emulsion comprises impregnating petrolatum into the substrate to produce a wound dressing having from about 5 wt % to about 75 wt % petrolatum.

15. The method of claim 13, wherein providing the substrate comprises hydroentangling polyester fibers to produce a nonwoven substrate, and wherein wherein introducing the oil emulsion comprises impregnating petrolatum into the nonwoven substrate to produce a wound dressing having from about 5 wt % to about 75 wt % petrolatum.

16. A method of preparing a wound dressing, comprising:
providing a fabric comprising cellulosic fibers woven with non-adherent polymeric fibers comprised of at least one polymer selected from the group consisting of polyester, polypropylene, polyethylene, and polytetrafluoroethylene;
impregnating an oil emulsion into the fabric to produce the wound dressing; and
sterilizing the wound dressing.

17. The method of claim 16, wherein providing the fabric comprises weaving at least a portion of the non-adherent polymeric fibers, in the weft direction, with at least a portion of the cellulosic fibers, in the warp direction, to produce a woven fabric with at least about 50% by weight of non-adherent polymeric fibers.

18. The method of claim 16, wherein providing the fabric comprises weaving at least a portion of the non-adherent polymeric fibers, in a warp direction, and at least a portion of the cellulosic fibers, in the weft direction, to produce a woven fabric with at least about 50% by weight of non-adherent polymeric fibers.

19. The method of claim 16, further comprising disposing the fabric into a sealable package, and wherein impregnating the oil emulsion comprises introducing petrolatum into the fabric disposed in the sealable package.

20. The method of claim 19, wherein providing the fabric comprises:
weaving the cellulosic fibers with the non-adherent polymeric fibers to produce a woven article;
bleaching the woven article;
tentering the bleached, woven article;
cutting the bleached, woven article to produce a woven fabric; and
folding the woven fabric to produce a gauze.

21. The method of claim 20, wherein folding the woven fabric comprises folding the woven fabric to produce the gauze having any of three plies, four plies, five plies, six plies, eight plies, ten plies, twelve plies, sixteen plies, 24 plies, 32 plies, 48 plies, 50 plies, 144 plies, and 216 plies.

22. The method of claim 16, further comprising introducing into the fabric at least one of a bacteriostatic agent and an antimicrobial agent.

23. The method of claim 16, wherein the non-adherent polymeric fiber is comprised of a material selected from the group consisting of polyethylene, polypropylene, polyfluoroethylene, polyfluoropropylene, polytetrafluoroethylene, polyethylene terephthalate, polyethylene naphthalate, polytrimethylene terephthalate, polybutylene terephthalate, and combinations thereof.
